(19) United States
(12) Patent Application Publication
(10) Pub. No.: US 2003/0219755 A1
(43) Pub. Date: Nov. 27, 2003

(54) Compositions and methods for performing hybridization assays using target enhanced signal amplification (TESA)

(76) Inventor: Nanibhushan Dattagupta, San Diego, CA (US)

Correspondence Address: Laurie A. Axford, Morrison & Foerster LLP, Suite 500, 3811 Valley Centre Drive, San Diego, CA, 92130, US

(21) Appl. No.: 10/155,666

(22) Filed: May 24, 2002

(51) Int. Cl. C12Q 1/70; C12Q 1/68

(52) U.S. Cl. 435/6; 435/5

(57) ABSTRACT

This invention relates to methods of signal amplification in nucleic acid hybridization reactions without the use of direct amplification of the target sequence. More particularly, it relates to methods of detecting target nucleic acids in samples such that detection is accomplished via probe-target and target-target hybridization. In one aspect, the present invention relates to methods of detecting genomic target nucleic acids such that the signal is amplified via formation of target-probe complexes.

### FIELD OF THE INVENTION

This invention relates to methods of signal amplification in nucleic acid hybridization reactions that permit the detection of low numbers of target nucleic acids. More particularly, the present invention relates to methods of detecting target nucleic acids in samples such that sensitivity is enhanced via complex target and probe interactions.

### BACKGROUND OF THE INVENTION

Nucleic acid hybridization reactions are commonly used in medical research and clinical diagnostics to detect the presence of target nucleic acid sequences that are associated with disease states or biological conditions. Generally, a nucleic acid probe is used that is capable of specifically hybridizing with the target sequence, and the amount of hybridization is detected using a variety of labeling techniques.

Detecting specific gene sequences in clinical samples is frequently hindered by the low copy number of these gene sequences in the sample. The ability to replicate these gene sequences to improve sensitivity has revolutionized modem molecular genetics. There are currently many different methods for amplifying nucleic acids in samples to improve assay sensitivity, such as: polymerase chain reaction (PCR) (U.S. Pat. Nos. 4,683,195 and 4,683,202); ligase chain reaction (LCR); nucleic acid sequence-based amplification (NASBA) (U.S. Pat. Nos. 5,409,818 and 5,554,517); strand displacement amplification (SDA); and transcription-medicated amplification (TMA).

Amplification methods are capable of providing more than one billion copies of a single target nucleic acid in a very short time. However, one of the principle problems of using amplification technologies is that they are susceptible to contamination by exogenous nucleic acids. These contaminating nucleic acids are then amplified along with the target nucleic acid in a clinical sample, which often leads to erroneous results. Methods that do not involve amplification suffer from a lack of sensivity in detecting target sequences that are present in a sample in limited amounts. Target amplification is useful to enhance sensitivity, but it has certain drawbacks associated with contamination, especially in clinical settings.

Another approach to improving assay sensitivity is to amplify the amount of detectable label that is associated with each copy of the target nucleic acid. This approach is referred to as “signal amplification”. One example of signal amplification involves the use of labeled linear or branched “enhancer” probes that bind to a portion of the probe-target hybrid to enhance the amount of detectable signal associated with each hybridization event. For example, Urdea et al. describe a method of signal amplification using branched oligonucleotide probes (U.S. Pat. Nos. 5,849,481 and 5,710,264.) However, such probes are difficult to synthesize and offer only a limited amplification by non-enzymatic methods, rendering this method cumbersome, expensive, and insufficiently sensitive for routine, clinical use.

A more complex amplification system is described by Segev (U.S. Pat. No. 5,846,709.) Chemically-modified probe pairs that differ in length are used to detect target nucleic acid. The two probes bind non-overlapping but closely situated regions of the same target, and a covalent linkage is formed between the two probes in proximity to one another after annealing to the target sequence. The target-probe hybrid is subsequently exposed to denaturing conditions, the newly formed probe is released, and the entire hybridization process is initiated again to permit another round of formation of covalently linked probes. Although additional cycling reduces spurious background signals, detection sensitivity is still a limiting factor.

The generation of multiple probes, requirement of multiple cycling steps, and the requirement of an additional detection steps make such methods difficult for ready application to the clinical diagnostic arena. Furthermore, these methods lack sufficient specificity and/or detectability to be useful for detecting low levels of analytes in heterogeneous samples.

In a clinical setting, the optimal diagnostic nucleic acid hybridization assay is one that has a limited number of steps, rapid detection time, and yet has sufficient sensitivity and specificity to make available diagnostic precision. Accordingly, it is an object of the present invention to provide a simple method of signal amplification in hybridization assay with a sensitivity that is particularly well adapted for use in detecting low numbers of target sequences in unpurified clinical samples.

### SUMMARY OF THE INVENTION

The present invention provides for methods of amplifying the detectable signal in a nucleic acid hybridization assay to increase the sensitivity such that low numbers of target nucleic acids are readily detectable. More specifically, these methods relate to the amplification of the detectable signal through formation of target-probe complexes.

In one aspect of the present invention, a method is provided for detecting a genomic target nucleic acid in a sample, comprising the steps of: a) contacting the genomic target nucleic acid with an immobilized nucleic acid probe that is complementary to a target sequence in the target nucleic acid under stringency conditions that permit target-probe hybridization and target-target hybridization to form a target-probe complex; b) contacting the target-probe complex with a detectable nucleic acid probe that hybridizes with the target nucleic acid but not with the target sequence to form a detectable target-probe network; and c) assessing target-probe hybridization by detecting the detectable probe. The detectable probe can be a genomic probe, and the method can also include a step of releasing target nucleic acid from cells if the sample is a cell-containing sample, such as with a lysis reagent. The method may also include a washing step to remove uncomplexed target nucleic acid and detectable probe.

Although the methods of the present invention are particularly well-suited for use with unpurified samples, they may also be used to assay purified samples or isolated target nucleic acids. In addition, the sample may be from, e.g., urine, blood, semen, cerebrospinal fluid, pus, amniotic fluid, tears, sputum, saliva, lung aspirate, vaginal discharge, urethral discharge, stool or biopsy specimens.

The methods may also be used to assay target nucleic acids from, e.g., bacterial or viral origin. For example, the assay may be designed to detect the presence of HIV, anthrax, or Mtb.

The method may also include the step of forming fragments of the target nucleic acid using enzymatic, mechanical or chemical digestion, to form fragments which may have an average length of between 500 and 5,000 nucleotides.

The immobilized probe can have any length, but is preferably between 10 and 50 nucleotides.

The detectable probe can also be any length and may be of synthetic or natural origin, but in one embodiment, the probe is a genomic probe, which may be either heterologous or homologous in origin when compared to the target nucleic acid, and may have an average length of between 500 and 5,000 nucleotides.

In one embodiment of the present invention, the immobilized probe is immobilized on a solid support, such as silicon, plastic, glass, ceramic, rubber, or polymer. The probe, or a library of probes, may be immobilized onto a biochip for simultaneous analysis of an array of different probes or samples.

The steps of the methods of the present invention can be performed in any sequence. For example, steps a) and b) can be performed simultaneously at a temperature such as 55° C. to about 90° C. In addition, a washing step can be added before step c).

The detectable label can be detected using any means for detecting hybridization reactions, such as when a detectable moiety such as a label is directly attached to the probe. For example, a detectable moiety can be attached for ever 50 nucleotides. The detectable moiety can be a label, such as a fluorophore, a chromophore, a lumiphore, a radioactive isotope, an electron dense moiety and a fluorescence resonance energy transfer moiety. Alternatively, the detectable probe may have an enzyme, a ligand or an enzyme substrate attached thereto. In addition, the detectable probe may have a label chemically linked thereto, such as acridine dyes, phenanthridines, phenazines, furocoumarins, phenothiazines and quinolines.

In an alternate embodiment, the detectable probe may have an intercalator compound bound thereto, such as mono-azido aminoalkyl methidium, mon-azido aminoalkyl ethidium, bis-azido aminoalkyl methidium, bis-azido aminoalkyl ethidium, ethidium monoazide, ethidium diazide, ethidium dimer azide, 4-azido-7-chloroquinoline, 2-azidofluorene, 4′-aminomethyl-4,5′-dimethylangelicin, 4′-aminomethyl-trioxsalen, 3-carboxy-5-amino-psoralen, 3-carboxy-8-amino-psoralen, 3-carboxy-5-hydroxy-psoralen and 3-carboxy-8-hydroxy-psoralen.

In yet another embodiment, the present invention provides for a kit for detecting a genomic target nucleic acid in an unpurified cell-containing sample that includes a nucleic acid probe immobilized on a solid surface that hybridizes with the target nucleic acid, a lysis reagent, and a heterogeneous detectable genomic probe that hybridizes with the target nucleic acid.

Other aspects of the present invention are described elsewhere herein.

### BRIEF DESCRIPTION OF THE DRAWINGS

FIG. 1 depicts the sequence of the IS6110 gene sequence from GenBank Accession Nos. Y15749 (Fang et al., J Clin. Microbiol., 181:1014-1020 (1999)), X17348 (Thierry et al., Nucleic Acids Res., 18 (1):188 (1990)) (SEQ. ID NO. 1)

FIG. 2 illustrates the method of the present invention for detection of genomic target nucleic acid using labeled genomic probe.

### DETAILED DESCRIPTION OF THE INVENTION

The present invention relates to compositions and methods for amplifying the signal in nucleic acid hybridization reactions to increase sensitivity. More particularly, it relates to hybridization assays between immobilized probes and target nucleic acids from samples. The assays are configured to promote target-target hybridization in addition to probe-target hybridization, which results in formation of a probe-target complex. Accordingly, such assays involve target enhanced sensitivity and are referred to as target enhanced signal amplification, or “TESA”.

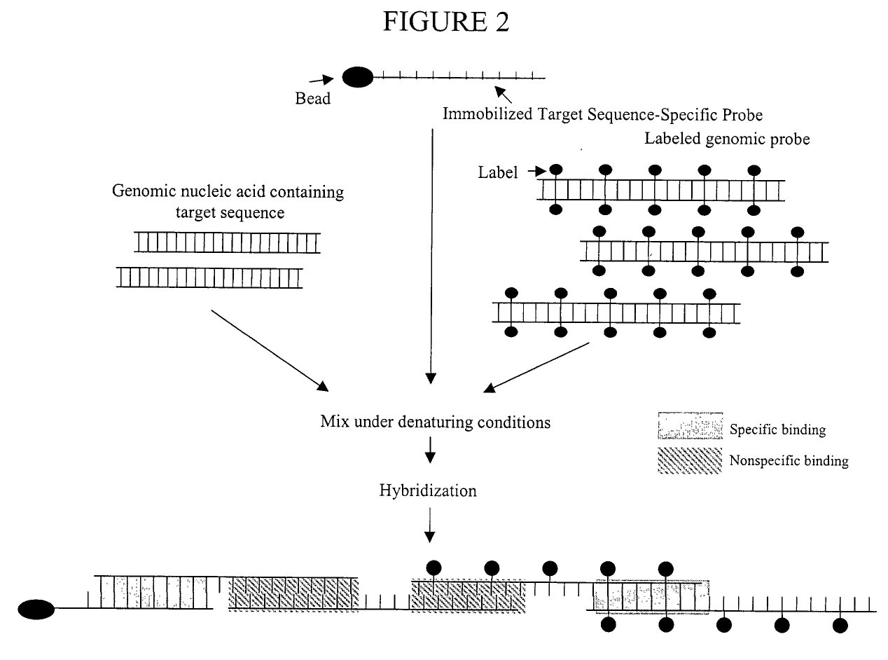

As shown in FIG. 2, in one embodiment of the present invention, the immobilized probe and the target nucleic acid hybridize to form a probe-target nucleic acid complex (not shown) that consists of both probe-target and target-target pairs, and either simultaneously or subsequently, the labeled genomic probe is introduced and binds to the target in the complex and also to itself (i.e. it forms intermolecular probe-probe hybrids) to form a labeled target-probe network (shown at the bottom of FIG. 2) that can easily be detected. By virtue of both specific hybridization (i.e., both “specific binding” and “nonspecific binding”) and intermolecular interactions, the signal is significantly enhanced.

The method of the present invention can generally be described in terms of the following steps, which may be performed sequentially or simultaneously as more fully described below: contacting the target nucleic acid with an immobilized probe such that both target-probe and target-target hybridization occurs to form a target-probe complex; b) contacting the target-probe complex with a detectable probe to form a detectable target-probe network; and c) assessing target-probe hybridization by detecting the detectable probe in the target-probe network.

Definitions

Unless defined otherwise, all technical and scientific terms used herein have the same meaning as is commonly understood by one of ordinary skill in the art to which this invention belongs. All patents, applications, published applications and other publications and sequences from GenBank and other databases referred to herein are incorporated by reference in their entirety. If a definition set forth in this section is contrary to or otherwise inconsistent with a definition set forth in the patents, applications, published applications and other publications and sequences from GenBank and other data bases that are herein incorporated by reference, the definition set forth in this section prevails over the definition that is incorporated herein by reference.

As used herein, “a” or “an” means “at least one” or “one or more.”

As used herein, “signal amplification” refers to a method for increasing the detectable signal for a particular target nucleic acid in a sample to improve assay sensitivity.

As used herein, “assessing” refers to quantitative and/or qualitative determination of the hybrid formed between the probe and the target nucleotide sequence, e.g., obtaining an absolute value for the amount or concentration of the hybrid, and also of obtaining an index, ratio, percentage, visual or other value indicative of the level of the hybrid. Assessment may be direct or indirect and the chemical species actually detected need not of course be the hybrid itself but may, for example, be a derivative thereof, reduction or disappearance of the probe and/or the target nucleotide sequence, or some further substance.

As used herein, “complementary” means that two nucleic acid sequences have at least 90% sequence identity. Preferably, the two nucleic acid sequences have at least 95%, 96%, 97%, 98%, 99% or 100% sequence identity. Alternatively, “complementary” means that two nucleic acid sequences can hybridize under high stringency condition(s).

As used herein, “genomic nucleic acid(s)” refers to the nucleic acids found within organisms, which can be cellular or acellular such as with viruses, and includes the nucleic acids in the nucleus as well as in organelles of eukaryotic organisms. Genomic nucleic acids when released and/or isolated from cells or viruses tend to become partially fragmented. Accordingly, genomic nucleic acids are different from synthetically produced or highly purified nucleic acids or products of amplification reactions, in that they are a heterogeneous population of nucleic acids with various sizes and sequences as opposed to a homogenous population of a single oligonucleotide.

As used herein, “hairpin structure” refers to a polynucleotide or nucleic acid that contains a double-stranded stem segment and a single-stranded loop segment wherein the two polynucleotide or nucleic acid strands that form the double-stranded stem segment are linked and separated by the single polynucleotide or nucleic acid strand that forms the loop segment. The “hairpin structure” can further comprise 3′ and/or 5′ single-stranded region(s) extending from the double-stranded stem segment.

As used herein, “heterologous” nucleic acid means that the source of the nucleic acid is different from the source of the target nucleic acid, while “homologous” nucleic acid means that the source of the nucleic acid is the same as the source of the target nucleic acid. For example, if the genomic target nucleic acid is from Mtb and the genomic detectable probe is from MOTT, the probe is “heterologous.”

As used herein, the term “label” refers to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

As used herein, “melting temperature” (“Tm”) refers to the midpoint of the temperature range over which nucleic acid duplex, i.e., DNA:DNA, DNA:RNA and RNA:RNA, is denatured. The Tm of the probe herein means the Tm of the hybridized probe.

As used herein, “Mycobacteria” refers to bacteria with unusual cell walls that are resistant to digestion, being waxy, very hydrophobic, and rich in lipid, especially esterified mycolic acids. Staining properties of Mycobacteria differ from those of Gram negative and Gram positive organisms, being acid-fast. Many Mycobacteria are intracellular parasites, causing serious diseases such as leprosy and tuberculosis. The Mycobacteria cell wall has strong immuno-stimulating adjuvant properties due to muramyl dipeptide (MDP).

As used herein, “Mycobacterium tuberculosis” (“Mtb”) refers to the bacterial species that is known to be one of the causative agents of the tuberculosis disease. Tuberculosis may affect almost any tissue or organ of the body, although it is most often found in the lungs. The anatomical lesion of tuberculosis is the tubercle, which can undergo caseation necrosis. Local symptoms of tuberculosis vary according to the body part affected. General symptoms of tuberculosis are those of sepsis, hectic fever, sweats, and emaciation. Tuberculosis is often progressive with high mortality if not treated.

As used herein, “nucleic acid(s)” refers to deoxyribonucleic acid (DNA) and ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with non-conventional phosphodiester backbones are also included herein, such as phosphotriester, polynucleopeptides, methylphosphonate, phosphorothioate, peptide-nucleic acids and the like.

As used herein, “probe” refers to an oligonucleotide that hybridizes to a target sequence, typically to facilitate its detection, but which also may serve as a primer. Unlike a primer that is used to prime the target nucleic acid in the amplification process, a probe need not be extended to amplify the target sequence using a polymerase enzyme. However, it will be apparent to those skilled in the art that probes and primers are structurally similar or identical in many cases.

As used herein, “target sequence” refers to a nucleic acid sequence within the target nucleic acid that is characteristic of or associated with the source of the target nucleic acid, and to which the probe specifically binds. As such, the target sequence is considered specific for a particular organism or cell type and can be targeted to identify an unknown organism or cell type or differentiate it from other organisms or cell types.

As used herein, a “probe does not contain any hairpin secondary structure” means that the nucleotide sequence in a probe that is heterologous to a target nucleotide sequence cannot form a hairpin structure in itself. However, the nucleotide sequence in a probe that is heterologous to a target nucleotide sequence can be a part of a probe that may form a hairpin structure under suitable conditions. For example, the nucleotide sequence heterologous to a target nucleotide sequence can be located within the loop or stem region of the hairpin probe, or can be located at the junction of the loop and stem region of the hairpin probe.

As used herein, “target-probe complex” refers to a complex of target and probe molecules, which includes both target-probe hybrids, as well as intermolecular target-target interactions.

As used herein, “detectable target-probe network” refers to a network of target sequence-specific probe, target nucleic acid and detectable probe molecules. Network formation may be precluded by complex formation, such as when the target nucleic acid is hybridized to the immobilized probe before introduction of the detectable probe. Alternatively, the network is formed directly by mixing together the immobilized probe, the target nucleic acid and the detectable probe simultaneously. In any event, the network consists at least three components: target sequence-specific probe-target nucleic acid hybrids, intermolecular target-target interactions and intermolecular detectable probe-detectable probe interactions.

As used herein: “stringency of hybridization” in determining percentage mismatch is as follows:

1) high stringency: 0.1×SSPE, 0.1% SDS, 65° C.;

2) medium stringency: 0.2×SSPE, 0.1% SDS, 50° C. (also referred to as moderate stringency); and

3) low stringency: 1.0×SSPE, 0.1% SDS, 50° C.

It is understood that equivalent stringencies may be achieved using alternative buffers, salts and temperatures (See generally, Ausubel (Ed.) Current Protocols in Molecular Biology, 2.9A. Southern Blotting, 2.9B. Dot and Slot Blotting of DNA and 2.10. Hybridization Analysis of DNA Blots, John Wiley & Sons, Inc. (2000)).

As used herein, the term “target nucleic acid” refers to the particular nucleic acid that a sample is suspected of containing. Such nucleic acids can be in biological samples, research materials, environmental samples, bodily fluids, and may be unpurified or purified using known methods. For an example of types of target analytes, see U.S. Pat. No. 5,792,614.

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein, “unamplified genomic nucleic acid” or “unamplifieid genomic sequence” means that total genomic DNA sequence has not been increased or amplified through means of replicating some or all of the DNA sequence using PCR, ligase chain reaction, etc.

Sample Preparation

The present invention provides methods of signal amplification in nucleic acid hybridization assays that permit the detection of low numbers of target nucleic acids without requiring amplification of the target nucleic acid itself. The assay system of the present invention is suitable for use with any sample suspected of containing target nucleic acids.

Any suitable samples, including samples of human, animal, or environmental (e.g., soil or water) origin, can be analyzed using the present method. Test samples can include body fluids, such as urine, blood, semen, cerebrospinal fluid, pus, amniotic fluid, tears, or semisolid or fluid discharge, e.g., sputum, saliva, lung aspirate, vaginal or urethral discharge, stool or solid tissue samples, such as a biopsy or chorionic villi specimens. Test samples also include samples collected with swabs from the skin, genitalia, or throat.

Test samples can be processed to release and/or isolate nucleic acid by a variety of means well known in the art (See generally, Ausubel (Ed.) Current Protocols in Molecular Biology, 2. Preparation and Analysis of DNA and 4. Preparation and Analysis of RNA, John Wiley & Sons, Inc. (2000)). It will be apparent to those skilled in the art that the sample suspected of containing target nucleic acids can be processed to release the nucleic acids from cells, organelles or viruses to expose them for binding to a probe, in which case the sample will also contain cells as well as cellular and subcellular debris (i.e. the nucleic acid is unpurified). As discussed elsewhere herein, the present invention is particularly well suited for use with unpurified samples.

Alternatively, the sample may be subjected to a method of isolating the nucleic acid in the sample that not only releases it from cells or subcellular structures, but also separates it from cellular and subcellular debris (i.e. the nucleic acid is purified.) Purified nucleic acids can be extracted from the aforementioned samples and may be measured spectraphotometrically or by other instrument for the purity.

If the source of target nucleic acid is cellular, a lysis reagent is preferably used prior to or simultaneously with the hybridization reaction. In a specific embodiment, samples are prepared for analysis in the present invention using the compositions, methods, and kits of Dattagupta, et al., U.S. Pat. No. 6,242,188 B1.

In a specific embodiment, a sample of human origin is assayed. In yet another specific embodiment, a sputum, urine, blood, tissue section, food, soil or water sample is assayed.

The Target Nucleic Acid

The present invention is useful for detecting a target nucleic acid sequence (“target sequence”), which may be all or a part of a target nucleic acid, that is associated with a particular organism or cell type. This include, inter alia, bacteria, viruses, fungi of human, animal or environmental origin. The method is also useful for detecting gene sequences or variants thereof that are associated with different animal cell types or disease states, such as malignancies and inherited diseases.

Preferably, the target sequence is associated with and thus useful for identifying the presence of infectious agents, including but not limited to Bacillus anthracis (anthrax), Clostridium botulinum, Yersinia pestis (plague), Variola major (smallpox), Francisella tularensis (tularmia), human immunodeficiency virus (AIDS), ebola virus, Marburg virus, arenaviruses, filoviruses, bunyaviruses, flaviviruses, Salmonella species, Vibrio cholerae, Brucella species, Clostridium perfringens, Burkholderia mallei (glanders), Coxiella burnetti (Q fever), Ricinus communis, Staphylococcus, hantaviruses, Mycobacterium tuberculosis, Nipah virus, tickbome encephalitis viruses, and yellow fever viruses. In a specific embodiment, the target nucleic acid is from Mycobacterium tuberculosis, and samples suspected of containing Mycobacterium organisms are assayed by the present invention. In another embodiment, the target nucleic acid is associated with HIV, and the sample analyzed by the present invention is one suspected of containing the HIV virus.

With all of the aforementioned methods for preparing a sample for hybridization of the target nucleic acid with the probe(s), it is to be understood that genomic target nucleic acids become at least partially digested during sample preparation. Accordingly, the target nucleic acid may have varying degrees of heterogeneity, depending on the nature of the sample and the process used to release and/or isolate the target nucleic acid. In addition, it may be desirable to deliberately promote formation of fragments from the genomic target nucleic acids by any known means to break nucleic acids, such as enzymatic, mechanical, and chemical digestion.

For example, when bacterial cells are lysed by heating in a sodium hydroxide solution, the genomic DNA is degraded to shorter lengths. Preferably, if the target sequence is genomic, it is partially digested. More preferably, the length of the genomic DNA is between 500 and 5,000 nucleotides, most preferrably with an average length of 1,000 nucleotides.

In one embodiment, the target nucleic acid is a heterogeneous genomic nucleic acid that is of cellular origin, in which case the method includes a step of releasing the nucleic acid from cells (and/or organelles), which necessarily results in not all of the genomic nucleic acid being in-tact (i.e. it is partially fragmented) during the hybridization reaction. Because of its heterogeneity, it is understood that not all target nucleic acid molecules will carry part or all of the target sequence.

When the target nucleic acid is a heterogeneous genomic nucleic acid that is double stranded, both the target sequence and its complement will be present during the hybridization reaction. As discussed herein, the hybridization reaction between probes and the target nucleic acid may be with either one or both of the complementary strands. For example, assays can easily be constructed which are the “mirror images” of the probe-target hybrids described herein and in the relevant literature by constructing probes that have sequences complementary to the probe sequences described herein and those described in the literature that hybridize with target sequences that are complementary to the target sequences described therein described.

Target Sequence-Specific Probes

The present invention provides for target sequence-specific probes for detecting a target nucleotide sequence. The probes can be in any suitable form. For example, the probe can comprise DNA, RNA, PNA or a derivative thereof. Alternatively, the probe can comprise both DNA and RNA or derivatives thereof. The probe can be single-stranded and be ready to be used in a hybridization analysis. Alternatively, the probe can be double-stranded and be denatured into single-stranded prior to the hybridization analysis.

The target sequence-specific probes can be produced by any suitable method. For example, the probes can be chemically synthesized (See generally, Ausubel (Ed.) Current Protocols in Molecular Biology, 2. 11. Synthesis and purification of oligonucleotides, John Wiley & Sons, Inc. (2000)), isolated from a natural source, produced by recombinant methods or a combination thereof. Synthetic oligonucleotides can also be prepared by using the triester method of Matteucci et al., J. Am. Chem. Soc., 3:3185-3191 (1981). Alternatively, automated synthesis may be preferred, for example, on a Applied Biosynthesis DNA synthesizer using cyanoethyl phosphoramidite chemistry. Preferably, the probes are chemically synthesized.

Suitable bases for preparing the target sequence-specific probes of the present invention may be selected from naturally occurring nucleotide bases such as adenine, cytosine, guanine, uracil, and thymine. It may also be selected from normaturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylpseudouridine, beta-D-galactosylqueosine, 2′-Omethylguanosine, inosine, N⁶-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N⁶-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N⁶-isopentenyladenosine, N-((9-.beta.-D-ribofuranosyl-2-methylthiopurine-6-yl)carbamoyl)threonine, N-((9-beta-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-end cap” strategy by which nuclease-resistant linkages are substituted for phosphodiester linkages at the 3′ end of the oligonucleotide (Shaw et al., Nucleic Acids Res., 19:747 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem., 36:1923 (1993)).

Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (MOMI) linkages. Phosphorothioate and methylphosphonate-modified oligonucleotides are particularly preferred due to their availability through automated oligonucleotide synthesis. The oligonucleotide may be a “peptide nucleic acid” such as described by (Milligan et al., J. Med. Chem., 36:1923 (1993)). The only requirement is that the oligonucleotide probe should possess a sequence at least a portion of which is capable of binding to a portion of the sequence of a target DNA molecule.

The target sequence-specific probes can be of any suitable length. There is no lower or upper limits to the length of the probe, as long as the probe hybridizes to the target nucleic acid and functions effectively as a probe (e.g., facilitates detection). The probes of the present invention can be as short as 50, 40, 30, 20, 15, or 10 nucleotides, or shorter. Likewise, the probes can be as long as 20, 40, 50, 60, 75, 100 or 200 nucleotides, or longer, e.g., to the full length of the target sequence. The target sequence-specific probe is preferably short in length with a probe length of not more than 100 nucleotides, more preferably with a length between 10 and 50 nucleotides, most preferably between 20 and 40 nucleotides.

The target sequence-specific probe preferably has have a G+C content ranging from about 50% to about 70%. More preferably, the probe has G+C content ranging from about 55% to about 65%. The probe should have a Tm value ranging from about 55° C. to about 90° C., preferably from about 65° C. to about 85° C.

The target sequence-specific probes used in the present invention are sufficiently complementary to the target sequence to form a stable hybrid therewith. The probes need not reflect the exact complementary sequence of the target sequence, but must be sufficiently complementary to hybridize selectively with the target sequence. Non-complementary bases or longer sequences can be interspersed into the probe, provided the probe retains sufficient complementarity with the target sequence to be hybridized and to thereby form a duplex structure which can be detected.

The target sequence-specific probe need not span the entire target sequence of interest. Any subset of the target sequence that has the potential to serve as a substrate for specific binding of the probe can be targeted. Consequently, the nucleic acid probe may hybridize to as few as 8 nucleotides of the target sequence. In addition, the target sequence-specific probe should be able to hybridize with a target sequence (or portion thereof) that is at least 8 nucleotides in length under low stringency. Preferably, the probe hybridizes with a target sequence of at least 8 nucleotides under middle or high stringency.

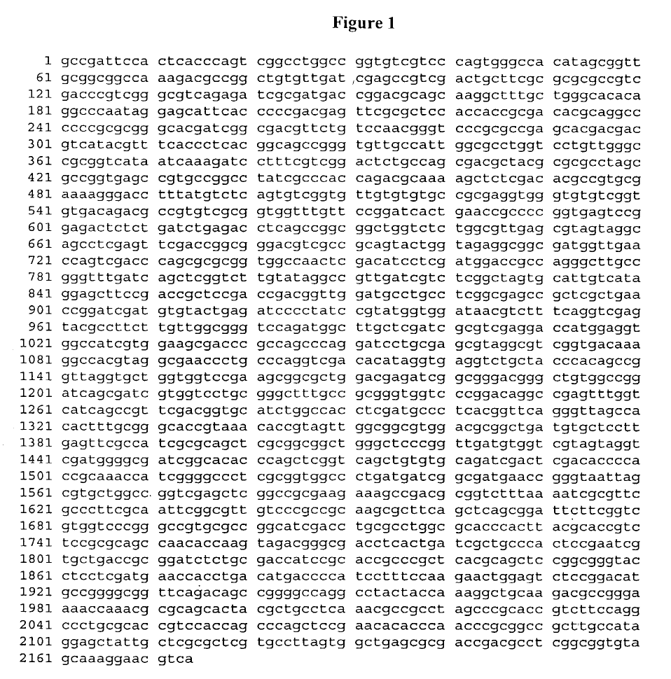

In a specific embodiment, the target sequence-specific probe is specific for Mycobacterium tuberculosis complex (TBC). In other words, it is capable of differentiating TBC from Mycobacteria Other Than Tuberculosis (MOTT), e.g., M. fortuitum, M. intracellulare, M. chelonae, M. scrofulaceum, and M. avium. Any suitable contiguous nucleotide sequence that is at least 8 nucleotides in length within Mycobacterium tuberculosis IS6110 (FIG. 1, Seq. ID. NO. 1) can be used as a target sequence-specifc probe.Exemplary Mtb IS6110 sequences include those disclosed in GenBank Accession Nos. Y15749 (Fang et al., J. Clin. Microbiol., 181:1014-1020 (1999)), X17348 (Thierry et al., Nucleic Acids Res., 18 (1):188 (1990)) and the nucleotide sequence of Mtb IS61110 sequence of strain CDC1551 (GenBank Accession # NC_{—}002755, 4.4 Mb complete sequence of CDC1551, in which there are several copies of IS6110, e.g., from nucleotide #3,115,312 to nucleotide #3,116,667).

Detectable Probes

The present invention can be practiced using either homogeneous probes or heterogeneous (e.g., genomic) probes that are detectable when hybridized with the target nucleic acid. The detectable probes are designed to form stable hybrids with the target nucleic acid, but not with the target nucleic acid sequence to which the target sequence-specific probe hybridizes. In other words, the detectable probes are designed so as to not compete with the target sequence-specific probe for binding to the target nucleic acid.

The design and preparation of detectable probes are well known in the field. As discussed in more detail elsewhere herein, the detectable probes may be detectable by attaching a label thereto that can be directly detected, or the detectable probe may be indirectly detected by attaching a chemical moiety to the probe, such as an enzyme, that causes or catalyzes a detectable event, such as conversion of an enzyme substrate to a detectable chemical species.

Preferably, the detectable probe is genomic nucleic acid that is isolated and prepared by any suitable method, including those described above under “Sample preparation” for preparing purified target nucleic acids. Other methods are well known in the art for purifying genomic nucleic acids from cellular or subcellular sources. In a preferred embodiment, the purified genomic nucleic acid is partially digested, either by means of the sample preparation itself, or by separate means as described above under “Sample preparation”.

Genomic nucleic acid probes are prepared to have sufficient complementarity with the target nucleic acid to form stable hybrids therewith, while having insufficient complementarity with the target sequence to compete with the target sequence-specific probe for binding to the target nucleic acid. In one embodiment, the detectable probe is derived from a species that is related to the species from whence the target nucleic acid came. For example, if the sample was suspected of containing Mtb, the detectable probe could be prepared from MOTT (i.e. a heterologous genomic nucleic acid), which contains a significant amount of genus-specific sequence homology with Mtb, but does not contain the target-specific sequences unique to Mtb to which the target sequence-specific probe binds.

Heterologous genomic nucleic acid sequences of species members within a particular genus are usually sufficiently complementary to each other to hybridize with one another. For example, it is well known that rRNA sequences of gram positive or gram negative bacteria have high complementarity, thus requiring a specific oligonucleotide probe to differentiate between species.

In an alternate embodiment, instead of using heterologous genomic nucleic acid, the detectable probe can be prepared from the same source as the target nucleic acid, such as from the same species of bacteria (i.e. homologous nucleic acid) from which the fragments bearing the complementary sequences to the target-specific sequences have been removed or prevented from binding to the target-specific sequence using known methods.

With either a heterologous or homologous detectable probe, the commercial advantages of the assays described herein are that the target nucleic acids do not have to be labeled, which would require an additional assay step, and the same detectable probe can be designed which would hybridize with nucleic acid from any member of a given taxonomic group and would thus be useful for multiple different assays.

Immobilization of Probes

The present method can be performed in solution. Preferably, it is conducted in chip format, e.g., by immobilizing the target sequence-specific probe(s) on a solid support.

The probes can be immobilized on any suitable surface, preferably a solid support, such as silicon, plastic, glass, ceramic, rubber, or polymer surface. The probe may also be immobilized in a 3-dimensional porous gel substrate, e.g., Packard HydroGel chip (Broude et al., Nucleic Acids Res., 29(19):E92 (2001)).

For an array-based assay, the probes are preferably immobilized to a solid support such as a “biochip”. The solid support may be biological, nonbiological, organic, inorganic, or a combination of any of these, existing as particles, strands, precipitates, gels, sheets, tubing, spheres, containers, capillaries, pads, slices, films, plates, slides, etc.

A microarray biochip containing a library of probes can be prepared by a number of well known approaches including, for example, light-directed methods, such as VLSIPS™ described in U.S. Pat. Nos. 5,143,854, 5,384,261 or 5,561,071; bead based methods such as described in U.S. Pat. No. 5,541,061; and pin based methods such as detailed in U.S. Pat. No. 5,288,514. U.S. Pat. No. 5,556,752, which details the preparation of a library of different double stranded probes as a microarray using the VLSIPS™, is also suitable for preparing a library of hairpin probes in a microarray.

Flow channel methods, such as described in U.S. Pat. Nos. 5,677,195 and 5,384,261, can be used to prepare a microarray biochip having a variety of different probes. In this case, certain activated regions of the substrate are mechanically separated from other regions when the probes are delivered through a flow channel to the support. A detailed description of the flow channel method can be found in U.S. Pat. No. 5,556,752, including the use of protective coating wetting facilitators to enhance the directed channeling of liquids though designated flow paths.

Spotting methods also can be used to prepare a microarray biochip with a variety of probes immobilized thereon. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much probe or other reagent as necessary at each stop. Typical dispensers include micropipettes, nanopippettes, ink-jet type cartridges and pins to deliver the probe containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously. Spotting methods are well known in the art and include, for example, those described in U.S. Pat. Nos. 5,288,514, 5,312,233 and 6,024,138. In some cases, a combination of flow channels and “spotting” on predefined regions of the support also can be used to prepare microarray biochips with immobilized probes.

A solid support for immobilizing probes is preferably flat, but may take on alternative surface configurations. For example, the solid support may contain raised or depressed regions on which probe synthesis takes place or where probes are attached. In some embodiments, the solid support can be chosen to provide appropriate light-absorbing characteristics. For example, the support may be a polymerized Langmuir Blodgett film, glass or functionalized glass, Si, Ge, GaAs, GaP, Sio₂, SiN₄, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art.

The surface of the solid support can contain reactive groups, which include carboxyl, amino, hydroxyl, thiol, or the like, suitable for conjugating to a reactive group associated with an oligonucleotide or a nucleic acid. Preferably, the surface is optically transparent and will have surface Si—OH functionalities, such as those found on silica surfaces.

The probes can be attached to the support by chemical or physical means such as through ionic, covalent or other forces well known in the art. Immobilization of nucleic acids and oligonucleotides can be achieved by any means well known in the art (see, e.g., Dattagupta et al., Analytical Biochemistry, 177:85-89(1989); Saiki et al., Proc. Natl. Acad. Sci. USA, 86:6230-6234(1989); and Gravitt et al., J. Clin. Micro., 36:3020-3027(1998)).

The probes can be attached to a support by means of a spacer molecule, e.g., as described in U.S. Pat. No. 5,556,752 to Lockhart et al., to provide space between the double stranded portion of the probe as may be helpful in hybridization assays. A spacer molecule typically comprises between 6-50 atoms in length and includes a surface attaching portion that attaches to the support. Attachment to the support can be accomplished by carbon-carbon bonds using, for example, supports having (poly)trifluorochloroethylene surfaces, or preferably, by siloxane bonds (using, for example, glass or silicon oxide as the solid support). Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful are surface attaching groups.

The spacer can also include an extended portion or longer chain portion that is attached to the surface-attaching portion of the probe. For example, amines, hydroxyl, thiol, and carboxyl groups are suitable for attaching the extended portion of the spacer to the surface-attaching portion. The extended portion of the spacer can be any of a variety of molecules which are inert to any subsequent conditions for polymer synthesis. These longer chain portions will typically be aryl acetylene, ethylene glycol oligomers containing 2-14 monomer units, diamines, diacids, amino acids, peptides, or combinations thereof.

In some embodiments, the extended portion of the spacer is a polynucleotide or the entire spacer can be a polynucleotide. The extended portion of the spacer also can be constructed of polyethyleneglycols, polynucleotides, alkylene, polyalcohol, polyester, polyamine, polyphosphodiester and combinations thereof. Additionally, for use in synthesis of probes, the spacer can have a protecting group attached to a functional group (e.g., hydroxyl, amino or carboxylic acid) on the distal or terminal end of the spacer (opposite the solid support). After deprotection and coupling, the distal end can be covalently bound to an oligomer or probe.

The present method can be used to analyze a single sample with a single probe at a time. Preferably, the method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously.

In another specific embodiment, the target sequence-specific probe is immobilized on a solid support adaptable to a single tube format. Preferably, the single tube format permits simultaneous contacting of the sample containing target nucleic acids with a mixture of reagents for cell lysis, immobilized target sequence-specific probe, and detectable probe under denaturing conditions.

Hybridization Conditions

Stringency conditions used in the present invention should permit specific hybridization of the target nucleic acid with the target sequence-specific immobilized probe with minimal, preferable negligible, nonspecific hybridization to the immobilized probe. In addition, it is an important feature of the present invention that he hybridization conditions permit target-target hybridization, which may be sequence specific or not. It is by virtue of these target-target interactions that the probe-target complex is formed. As such, each target sequence-specific probe molecule is capable of forming a complex with multiple target nucleic acid molecules. This is the essence of the “target enhanced signal amplification” (TESA) that is observed with the present invention.

Accordingly, the conditions of hybridization should be such that hybridization with the target sequence-specific probe and intramolecular hybridization of the target nucleic acids occur. This is invariably the case when the target nucleic is genomic nucleic acid. It is preferable to have the mixture of target nucleic acid and target sequence-specific probe mixed together under denaturing conditions before the mixture is incubated under hybridization conditions. Because hybridization conditions are selected for specific hybridization between target nucleic acid and target sequence-specific probe, the remaining genomic nucleic acid molecules that do not hybridize with the probe will produce both specific and non-specific hybrids that result in a multi-molecular aggregate of nucleic acids, i.e. a probe-target complex is formed.

Preferably, the probe-target hybrid has a Tm under stringency conditions that prevents nonspecific hybridization hybridization with the probe, but does not prevent nonspecific target-target hybridization. For example, the Tm for a 20-mer oligonucleotide hybridizing to its complementary target can be 60° C. while the corresponding Tm for 1 kB genomic probe with several mismatches will be higher, thus permitting nonspecific hybridization.

Hybridization can be carried out under any suitable conditions known in the art. It will be apparent to those skilled in the art that hybridization conditions can be altered to increase or decrease the degree of hybridization, the level of specificity of the hybridization, and the background level of non-specific binding (i.e., by altering hybridization or wash salt concentrations or temperatures). The hybridization between the probe and the target nucleotide sequence can be carried out under any suitable stringencies, including high, middle or low stringency. Typically, hybridizations will be performed under conditions of high stringency.

The hybridization can be carried out at any suitable temperature. For example, if the present probe is part of a hairpin structure, the oligonucleotide probe and the target nucleotide sequence can be contacted at a temperature from about 4° C. to about 90° C. Preferably, the oligonucleotide probe and the target nucleotide sequence can be contacted at a temperature from about 55° C. to about 90° C., preferably from about 65° C. to about 85° C.

In addition, the hybridization can be carried out for any suitable period of time. For example, if the present probe is part of a hairpin structure as disclosed in co-owned PCT Patent Application No. WO 02/106531, the oligonucleotide probe and the target nucleotide sequence can be contacted for a time from about 1 minute to about 60 minutes. Preferably, the oligonucleotide probe and the target nucleotide sequence can be contacted for a time from about 15 minutes to about 30 minutes.

Conditions that affect hybridization and that select against nonspecific hybridization are also known in the art (Molecular Cloning A Laboratory Manual, second edition, J. Sambrook, E. Fritsch, T. Maniatis, Cold Spring Harbor Laboratory Press, 1989). Generally, lower salt concentration and higher temperature increase the stringency of hybridization. For example, in general, stringent hybridization conditions include incubation in solutions that contain approximately 0.1×SSC, 0.1% SDS, at about 65° C. incubation/wash temperature. Middle stringent conditions are incubation in solutions that contain approximately 1-2XSSC, 0.1% SDS and about 50° C.-65° C. incubation/wash temperature. The low stringency conditions are 2×SSC and about 30° C.-50° C.

An alternate method of hybridization and washing is first to carry out a low stringency hybridization (5×SSPE, 0.5% SDS) followed by a high stringency wash in the presence of 3M tetramethyl-ammonium chloride (TMAC). The effect of the TMAC is to equalize the relative binding of A-T and G-C base pairs so that the efficiency of hybridization at a given temperature corresponds more closely to the length of the polynucleotide. Using TMAC, it is possible to vary the temperature of the wash to achieve the level of stringency desired (Wood et al., Proc. Natl. Acad. Sci. USA, 82:1585-1588 (1985)).

A hybridization solution may contain 25% formamide, 5×SSC, 5× Denhardt's solution, 100 μg/ml of single stranded DNA, 5% dextran sulfate, or other reagents known to be useful for probe hybridization.

Detecting Target Sequence-Specific Probe-Target Hybridization

The presence and/or amount of hybridization between the target sequence-specific probe and the target nucleic acid is assessed to determine the characteristics of the source of target nucleic acid. As discussed elsewhere herein, by selecting a target sequence that is characteristic of a particular bacterial species or virus type, an unknown sample can be assayed to determine if this bacterial species or viral type is present in the sample.

Formation of the probe-target complex necessarily requires specific hybridization between the target sequence-specific probe and the target nucleic acid. Accordingly, the degree of complex formation is directly related to the degree of specific probe-target hybridization, and can be assessed by adding a detectable probe that hybridizes to the target nucleic acid in the probe-target complex.

Preferably, the detectable probe is labeled genomic nucleic acid. The genomic nucleic acid source can be homologous or heterologous. If the genomic nucleic acid is heterologous to the target nucleic acid, the amount of sequence complementarity between the target nucleic acid and the detectable probe should be at least 20%, preferably between 30 and 99%, most preferably between 95 and 99%. Detection of hybridization between the detectable probe and the target nucleic acid can be carried out by any method known in the art, e.g., labeling heterologous genomic probe, homologous genomic probe, the target genomic nucleic acids or some combination thereof. In addition, the probe is considered “detectable” if it hybridizes with the target nucleic acid in the probe-target complex sufficiently to be detected by mass spectroscopy in the absence of a directly detectable label. (See, e.g., U.S. Pat. No. 6,300,076).

In a specific embodiment of the invention, the labeling method provides about 1 detectable label moiety per 50 nucleotides of the detectable probe. Using a genomic detectable probe with an average fragment size of 1,000 nucleotides, such labeling results in 20 detectable label moieties per fragment.

The detectable label is a moiety that can be detected either directly or indirectly after hybridization. In other words, a detectable label has a measurable physical property (e.g., fluorescence or absorbance) or is participant in an enzyme reaction. Using direct labeling, the probe is labeled, and the formation of labeled probe-target hybrids is assessed by detecting the label bound to the probe-target complex. Using indirect labeling, another chemical moiety is introduced that is capable of being detected in proportion to the amount of detectable probe-target hybrid formation, which in turn is proportional to the amount of target sequence-specific probe-target nucleic acid hybridization.

Methods of labeling probes are well known in the art. Suitable labels include fluorophores, chromophores, luminophores, radioactive isotopes, electron dense reagents, FRET(fluorescence resonance energy transfer), enzymes and ligands having specific binding partners. Particularly useful labels are enzymatically active groups such as enzymes (Wisdom, Clin. Chem., 22:1243 (1976)); enzyme substrates (British Pat. No. 1,548,741); coenzymes (U.S. Pat. Nos. 4,230,797 and 4,238,565) and enzyme inhibitors (U.S. Pat. No. 4,134,792); fluorescers (Soini and Hemmila, Clin. Chem., 25:353 (1979)); chromophores including phycobiliproteins, luminescers such as chemiluminescers and bioluminescers (Gorus and Schram, Clin. Chem., 25:512 (1979) and ibid, 1531); specifically bindable ligands, i.e., protein binding ligands; antigens; and residues comprising radioisotopes such as ³H, ³⁵S, ³²P, ¹²⁵I, and ¹⁴ C. Such labels are detected on the basis of their own physical properties (e.g., fluorescers, chromophores and radioisotopes) or their reactive or binding properties (e.g., antibodies, enzymes, substrates, coenzymes and inhibitors). Ligand labels are also useful for solid phase capture of the oligonucleotide probe (i.e., capture probes). Exemplary labels include biotin (detectable by binding to labeled avidin or streptavidin) and enzymes, such as horseradish peroxidase or alkaline phosphatase (detectable by addition of enzyme substrates to produce a colored reaction product).

For example, a radioisotope-labeled probe can be detected by autoradiography. Alternatively, a probe labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As a further alternative, the probe may be labeled with a moiety that requires additional reagents to detect hybridization. If the label is an enzyme, the labeled nucleic acid, e.g., DNA, is ultimately placed in a suitable medium to determine the extent of catalysis. For example, a cofactor-labeled nucleic acid can be detected by adding the enzyme for which the label is a cofactor and a substrate for the enzyme. Thus, if the enzyme is a phosphatase, the medium can contain nitrophenyl phosphate and one can monitor the amount of nitrophenol generated by observing the color. If the enzyme is a beta-galactosidase, the medium can contain o-nitro-phenyl-D-galacto-pyranoside, which also liberates nitrophenol. Examples of the latter include, but are not limited to, beta-galactosidase, alkaline phosphatase, papain and peroxidase. For in situ hybridization studies, the final product of the substrate is preferably water insoluble. Other labels, e.g., dyes, will be evident to one having ordinary skill in the art.

The label can be linked directly to the DNA binding ligand, e.g., acridine dyes, phenanthridines, phenazines, furocoumarins, phenothiazines and quinolines, by direct chemical linkage such as involving covalent bonds, or by indirect linkage such as by the incorporation of the label in a microcapsule or liposome, which in turn is linked to the binding ligand. Methods by which the label is linked to a DNA binding ligand such as an intercalator compound are well known in the art and any convenient method can be used. Representative intercalating agents include mono-or bis-azido aminoalkyl methidium or ethidium compounds, ethidium monoazide, ethidium diazide, ethidium dimer azide (Mitchell et al., J. Am. Chem. Soc., 104:4265 (1982))), 4-azido-7-chloroquinoline, 2-azidofluorene, 4′-aminomethyl-4,5′-dimethylangelicin, 4′-aminomethyl-trioxsalen (4′aminomethyl-4,5′,8-trimethyl-psoralen), 3-carboxy-5- or -8-amino- or -hydroxy-psoralen. A specific nucleic acid binding azido compound has been described by Forster et al., Nucleic Acid Res., 13:745 (1985). Other useful photoreactable intercalators are the furocoumarins which form (2+2) cycloadducts with pyrimidine residues. Alkylating agents also can be used as the DNA binding ligand, including, for example, bis-chloroethylamines and epoxides or aziridines, e.g., aflatoxins, polycyclic hydrocarbon epoxides, mitomycin and norphillin A. Particularly useful photoreactive forms of intercalating agents are the azidointercalators. Their reactive nitrenes are readily generated at long wavelength ultraviolet or visible light and the nitrenes of arylazides prefer insertion reactions over their rearrangement products (White et al., Meth. Enzymol., 46:644 (1977)).

The probe may also be modified for use in a specific format such as the addition of 10-100 T residues for reverse dot blot assays or the conjugation to bovine serum albumin or immobilization onto magnetic beads.

The detectable probe can be added after hybridization between the target sequence-specific probe and the target nucleic acid (the target-specific hybridization reaction) or it may be present during the target-specific hybridization reaction. Optionally, the hybridization conditions may be modified after addition of the detectable probe. After the target-specific hybridization reaction, unhybridized target nucleic acid can be separated from the target-probe network, for example, by washing if the target-probe network is immobilized. In the case of a solid support such as a biochip, detection of label bound to locations on the support indicates hybridization of the target sequence in the sample to the target sequence-specific probe.

The detectable probe can be labeled heterologous genomic probe consisting of a mixture of whole genomic DNA essentially as described in U.S. Pat. No. 5,348,855. Labeling can be accomplished with intercalating dyes if the probe contains double stranded DNA. Preferred DNA-binding ligands are intercalator compounds such as those described above.

Advantageously, the DNA binding ligand is first combined with label chemically and thereafter combined with the nucleic acid probe. For example, since biotin carries a carboxyl group, it can be combined with a furocoumarin by way of amide or ester formation without interfering with the photochemical reactivity of the furocoumarin or the biological activity of the biotin. Aminomethylangelicin, psoralen and phenanthridium derivatives can similarly be linked to a label, as can phenanthridium halides and derivatives thereof such as aminopropyl methidium chloride (Hertzberg et al, J. Amer. Chem. Soc., 104:313 (1982)). Alternatively, a bifunctional reagent such as dithiobis succinimidyl propionate or 1,4-butanediol diglycidyl ether can be used directly to couple the DNA binding ligand to the label where the reactants have alkyl amino residues, again in a known manner with regard to solvents, proportions and reaction conditions. Certain bifunctional reagents, possibly glutaraldehyde may not be suitable because, while they couple, they may modify nucleic acid and thus interfere with the assay. Routine precautions can be taken to prevent such difficulties.

Also advantageously, the DNA binding ligand can be linked to the label by a spacer, which includes a chain of up to about 40 atoms, preferably about 2 to 20 atoms, including, but not limited to, carbon, oxygen, nitrogen and sulfur. Such spacer can be the polyfunctional radical of a member including, but not limited to, peptide, hydrocarbon, polyalcohol, polyether, polyamine, polyimine and carbohydrate, e.g., -glycyl-glycyl-glycyl- or other oligopeptide, carbonyl dipeptides, and omega-amino-alkane-carbonyl radical or the like. Sugar, polyethylene oxide radicals, glyceryl, pentaerythritol, and like radicals also can serve as spacers. Spacers can be directly linked to the nucleic acid-binding ligand and/or the label, or the linkages may include a divalent radical of a coupler such as dithiobis succinimidyl propionate, 1,4-butanediol diglycidyl ether, a diisocyanate, carbodiimide, glyoxal, glutaraldehyde, or the like.

Quantitative hybridization assays can also be performed according to the present invention. The amount of detectable probe bound to a microarray spot can be measured and can be related to the amount of target nucleic acid which is in the sample. Dilutions of the sample can be used along with controls containing known amount of the target nucleic acid. The precise conditions for performing these steps will be apparent to one skilled in the art. In microarray analysis, the detectable label can be visualized or assessed by placing the probe array next to x-ray film or phosphoimagers to identify the sites where the probe has bound. Fluorescence can be detected by way of a charge-coupled device (CCD) or laser scanning.

Kits

The present invention further provides for the packaging of components for the hybridization assay in a kit format, preferably with instructions for using the probes to detect a target nucleic acid in a sample. The components of the kit may be packaged together in a common container, typically including written instructions for performing selected specific embodiments of the methods disclosed herein. Such components can include, for example, cell lysis reagents, target sequence-specific probe preferably immobilized on a solid support, detectable (preferably genomic) probe, and/or reagents and means for assessing hybrid formation (e.g., radiolabel, enzyme substrates, antibodies, etc., and the like).

### EXAMPLES

### Example 1

### Selection of IS6110 Probes for TBC Detection

Probes were designed that would hybridize with the IS6110 element with high specificity and sensitivity in a way to avoid false positive results. The BLASTN 2.1.3 program (available online at NCBI web site) was used to search all nucleic acid databases in GenBank using GenBank Accession # Y15749 (IS6110) (FIG. 1, Seq. ID No. 1) as a query sequence with either multiple or pair wise alignments, and with relatively less permissive conditions. Various sub-regions of the IS6110 that appeared to be conservative among the TBC were identified as potential sites for designing probes. After an intensive analysis for hybridization characteristics, secondary hairpin structure and thermal profiles, several probes were selected from different sub-regions that were highly specific to IS6110. The sequences of these potential probes were then used as query sequences and compared to all nucleic acid databases in GenBank. The probes having significant identity with sequences of other genes in different organisms were eliminated and three probes were analyzed for their base composition, G+C content, thermal stability, hybridization characteristics and absence of hairpin structure formation. They are as follows:

| | |
|---|---|
| AGT01012: | |
| ¹TTCCGACCGCTCCGACCGACGGTTG²⁵ | (SEQ ID NO:2) |
| AGT01014: | |
| ¹CATCAGCCGTTCGACGGTGCATCTG²⁵ | (SEQ ID NO:3) |
| AGT01041: | |
| ¹ACCGCTCCGACCGACGGTT¹⁹ | (SEQ ID NO:4) |

The sequence of the probe AGT01012 starts at nt #846 and ends at nt #870 of the GenBank Accession # Y15749 (IS6110) and gave 53 Blast hits, all of which had very good (low) E values (2e-05) for TBC. The sequence of the probe AGT01014 starts at nt #1261 and ends at nt #1285 of the GenBank Accession # Y15749 (IS6110) and gave 51 Blast hits, all but three of which had very good E values of 2e-05 for TBC. Two hits had high and non-significant E values of 0.36 for N. meningitidis whereas the remaining hit had low E value of 0.09 for Mtb. AGT01041 is a 19 mer sequence that is nested in AGT01012.

### Example 2

### Demonstration of Specificity of Probes

The following example is to demonstrate specificity of the probes AGT 01012, 01014 and 01041. The experiments were carried out by labeling genomic nucleic acid samples with a photo-chemically activatable compound. The labeled nucleic acid samples were then hybridized with the probes which were chemically immobilized to magnetic particles. The hybridized materials were detected by chemiluminescence of the label.

More particularly, clinical isolates were cultured from patients who were diagnosed with TB. Nucleic acids were isolated from the clinical isolates by lysing cells with Triton X-100 in a Tris-EDTA buffer. Further purification of the nucleic acid was carried out by phenol-chloroform extraction and ethanol precipitation. The precipitated DNA was dissolved in water (1 mg/ml).

The labeling compound was synthesized by the methods described in Dattagupta et al., U.S. Pat. No. 6,242,188 B1. The compound APA was synthesized by following the procedure described in Example 17 of U.S. Pat. No. 6,242,188 except in step 5 of the synthesis, methyl flurosulfonate succinimdo acridine (described in Example 19, step 3) was used instead of biotin compound. The compound was dissolved in ethanol (10 mg/ml).

10 μl of APA was added to 100%1 of DNA solution (1 mg/ml). The solution was irradiated for 20 minutes using a hand held long wavelength (365 nm) UV lamp at room temperature. After the labeling reaction, excess unbound APA was removed by ethanol precipitation. The labeled sample was then hybridized with immobilized probes at 83° C. for 10 minutes in hybridization buffer (100 mM NaCl, 3% triton X-102, 50 mM PIPES, pH 6.5) and washed 4 times using the wash buffer (20 mM NaCl, 3% triton X-102, 50 mM PIPES, pH 6.5) at the hybridization temperature. The hybrid was detected by chemiluminescence from acridinium ester label in a commercial luminometer (Zylux, Maryville, Tenn.).

APA-labeled positive pool of DNA from Mtb and APA-labeled DNA from each of the five MOTT species (M. fortuitum, M. intracellulare, M. chelonae, M scrofulaceum and M. avium), were used to test specificity of AGT01012 (FIG. 1), AGTO01014 (FIG. 2) and AGT01041 (FIG. 3). The positive pool of DNA was a mixture of an equal amount of DNA from five different clinical isolates of Mtb. The results indicate that all three probes differentiate Mtb from MOTT very well.

### Example 3

### Assessment of Probe-Target Complex Formation

The following example demonstrates the enhancement of the target-to-probe ratio when the target is genomic nucleic acid using labeled target nucleic acid as described above in Example 2.

Mycobacterium tuberculosis are lysed by heating in sodium hydroxide solution. As a result, the genomic DNA in the bacterial cells is degraded to shorter lengths, generating genomic fragments. The average length of the genomic fragments is 1,000 nucleotides, with a range of length between 500 and 5,000 nucleotides.

The genomic fragments prepared above are labeled with an estimated maximum efficiency of 1 detectable moiety per 50 base pairs, or approximately 20 detectable moieties per genomic fragment. In the absence of signal amplification, the signal from such fragments would be below detectable levels.

In a sample container, immobilized target sequence-specific probes as described above in Example 1 and reagents for cell lysis are premixed with the labeled genomic target nucleic acid and incubated at 100° C. to denature the nucleic acid, and then incubated at a temperature selected to maximize hybridization efficiency so as to permit specific hybridization with the immobilized probe and target-target hybridization between target nucleic acid fragments, i.e. formation of probe-target complexes. Target nucleic acid that is free in solution is removed by washing the immobilized probe-target complex.

Nanogram quantities of genomic Mtb are detectable following hybridization. The Mtb genome is approximately 4,400,000 basepairs. With a 1,000 base pair fragment carrying the target sequence, about 1/4000th of the DNA should produce a signal due to hybridization in the absence of any target-target hybridization mediated signal amplification. In other words, from 8.8 nanograms of double-stranded genomic DNA, only one picogram equivalent of signal generating hybrid would be produced. With an approximate average molecular weight of 660 Daltons per base pair, a picogram of DNA fragments of 1,000 base pair length has approximately 1.5 attomoles of target analyte producing about 100 Relative Light Units (RLUs), assuming 100% hybridization efficiency. However, the target nucleic acid is detectable in the range of 100,000 RLUs, reflecting the degree of complex formation between target molecules and the immobilized probe.

The extent of signal amplification indicative of the degree of probe-target complex formation is at least 800-fold that which would be expected in the absence of target-target interactions. The measured value of chemiluminescence signal using the labeling compound demonstrates 1.6×.10¹⁰ molecules of label produce 100,000 RLUs of detectable chemiluminescence signal. Assuming there are 20 labels per 1,000 basepair genomic probe, 8×10⁸ molecules of DNA are detected while only 10⁶ copies of hybridizable DNA is present in a picogram of DNA. This reflects an 800-fold amplification in signal, which indicates that probe-target complex formation will significantly enhance sensitivity in detecting sequences in low copy number using unlabeled target nucleic acid and labeled probe as described in Example 4.

In summary, this experiment demonstrates that the number of target molecules associated with each probe molecule can be enhanced, which in turn provides a platform for enhancing signal using a detectable probe that binds to the target nucleic acids in the complex.

### Example 4

### Detection of TBC using IS6110 Probe

The following example demonstrates the detection of Mycobacterium tuberculosis using immobilized IS6110-specific probe and labeled genomic probe from Mycobacterium fortuitum. The assay format is also depicted in FIG. 2. In this example, the Mycobacterium fortuitum probe is serving as a detectable heterologous genomic nucleic acid probe, and would be useful as the detectable probe in any assay for Mycobacterium species.

Clinical isolates were cultured from patients who were diagnosed with TB (test patients), and also from patients known not to have TB (control patients) as described in example 2.

Mycobacterium fortuitum nucleic acid was cultured and isolated as described for clinical isolates and labeled as described in example 2.

The IS6110 probe (Seq. ID NO.2) was immobilized chemically to magnetic particles. In a sample container, the labeled genomic Mycobacterium fortuitum probe, the unlabeled genomic target nucleic acid (from either the test patients or control patients) and the immobilized IS6110 probe were mixed together and allowed to hybridize as described in Example 2.

The immobilized labeled probe-target complex was washed in hybridization buffer at hybridization temperature four times. Chemiluminescence was detected as described in Example 2. Mtb was easily detectable from the test patient isolates, which demonstrated an average chemiluminescence of 100,428 RLUs, while the control patient isolated demonstrated an average chemiluminescence of 6,544 RLUs.

As shown in FIG. 2, when using labeled genomic probe to detect genomic target nucleic acid, there are two sources of amplification—one stems from target-target hybridization and the other stems from intermolecular (i.e. probe-probe) hybridization. Accordingly, the method of the present invention can be used to significantly enhance sensitivity of assays for detection of low copy number target nucleic acids. As described herein, all steps of the assay method may be performed simultaneously or sequentially, depending on the nature of the assay format.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A method for detecting a genomic target nucleic acid in a sample, comprising the steps of:
a) contacting the genomic target nucleic acid with an immobilized nucleic acid probe that is complementary to a target sequence in the target nucleic acid under stringency conditions that permit target-probe hybridization and target-target hybridization;
b) simultaneously or subsequently contacting the genomic target nucleic acid with a detectable nucleic acid probe that hybridizes with the target nucleic acid but not with the target sequence to form a detectable target-probe network; and
c) assessing target-probe hybridization by detecting the detectable probe.

2. The method of claim 1, wherein the detectable probe is a genomic probe.

3. The method of claim 1, further comprising the step of releasing the target nucleic acid from cells in the sample.

4. The method of claim 3, wherein the step of releasing the target nucleic acid comprises contacting the cells with a lysis reagent.

5. The method of claim 1, further comprising the step of isolating the target nucleic acid from the sample.

6. The method of claim 1, wherein the sample further comprises cellular debris.

7. The method of claim 1, wherein the sample is selected from the group consisting of: urine, blood, semen, cerebrospinal fluid, pus, amniotic fluid, tears, sputum, saliva, lung aspirate, vaginal discharge, urethral discharge, stool or biopsy specimens.

8. The method of claim 1, wherein the target nucleic acid is from a bacterial or viral infectious agent.

9. The method of claim 8, wherein the infectious agent is human immunodeficiency virus.

10. The method of claim 8, wherein the infectious agent is Bacillus anthracis.

11. The method of claim 8, wherein the infectious agent is Mycobacterium tuberculosis.

12. The method of claim 1, further comprising the step of forming fragments of the target nucleic acid using enzymatic, mechanical or chemical digestion.

13. The method of claim 1, wherein the fragments have an average length of between 500 and 5,000 nucleotides.

14. The method of claim 1, wherein the immobilized probe has a length of between 10 and 50 nucleotides.

15. The method of claim 1, wherein the detectable probe is a detectable genomic probe.

16. The method of claim 15, wherein the detectable genomic probe is homologous with the target nucleic acid.

17. The method of claim 15, wherein the detectable genomic probe is heterologous with the target nucleic acid.

18. The method of claim 15, wherein the genomic probe comprises fragments having an average length of between 500 and 5,000 nucleotides.

19. The method of claim 1, wherein the immobilized probe is immobilized on a solid support.

20. The method of claim 1, wherein the solid support is selected from the group consisting of silicon, plastic, glass, ceramic, rubber, or polymer.

21. The method of claim 1, wherein the probe is immobilized to a biochip.

22. The method of claim 1, wherein steps a) and b) are performed simultaneously at a temperature of between 55° C. to about 90° C.

23. The method of claim 1, further comprising a step of washing the detectable target-probe network formed in step b) prior to step c).

24. The method of claim 1, wherein the detectable probe is labeled with a detectable moiety.

25. The method of claim 24, wherein the detectable probe bears at least one detectable moiety per every 50 nucleotides.

26. The method of claim 24, wherein the detectable moiety is a label selected from the group consisting of: a fluorophore, a chromophore, a lumiphore, a radioactive isotope, an electron dense moiety and a fluorescence resonance energy transfer moiety.

27. The method of claim, 1, wherein the detectable probe further comprises an enzyme, a ligand or an enzyme substrate attached thereto.

28. The method of claim 1, wherein the detectable probe has a label chemically linked directly thereto selected from the group consisting of: acridine dyes, phenanthridines, phenazines, furocoumarins, phenothiazines and quinolines.

29. The method of claim 1, wherein the detectable probe has an intercalator compound bound thereto selected from the group consisting of: mono-azido aminoalkyl methidium, mon-azido aminoalkyl ethidium, bis-azido aminoalkyl methidium, bis-azido aminoalkyl ethidium, ethidium monoazide, ethidium diazide, ethidium dimer azide, 4-azido-7chloroquinoline, 2-azidofluorene, 4′-aminomethyl-4,5′-dimethylangelicin, 4′-aminomethyl-trioxsalen, 3-carboxy-5-amino-psoralen, 3-carboxy-8-amino-psoralen, 3-carboxy-5-hydroxy-psoralen and 3-carboxy-8-hydroxy-psoralen.

30. A method for detecting a genomic target nucleic acid in a sample, comprising the steps of:
a) contacting the genomic target nucleic acid with an immobilized nucleic acid probe that is complementary to a target sequence in the target nucleic acid under stringency conditions that permit target-probe hybridization and target-target hybridization to form a target-probe complex;
b) contacting the target-probe complex with a detectable nucleic acid probe that hybridizes with the target nucleic acid but not with the target sequence to form a detectable target-probe network; and
c) assessing target-probe hybridization by detecting the detectable probe.

31. A kit for detecting a genomic target nucleic acid in an unpurified cell-containing sample, comprising:
a) a nucleic acid probe immobilized on a solid surface that hybridizes with the target nucleic acid to form target-probe complexes;
b) a lysis reagent; and
c) a heterogeneous detectable genomic probe that hybridizes with the target nucleic acid in the target-probe complex.
